(19) United States
(12) Patent Application Publication
(10) Pub. No.: US 2011/0208045 A1
(43) Pub. Date: Aug. 25, 2011

(54) Marking Clip for an Area of the Inside Wall of the Stomach or Intestine

(76) Inventor: Cora Wex, Markisch Luch (DE)

(21) Appl. No.: 13/057,022

(22) PCT Filed: Jul. 30, 2009
(86) PCT No.: PCT/EP09/59858

§ 371 Date: Apr. 30, 2011

(30) Foreign Application Priority Data
Aug. 1, 2008 (DE) 10 2008 037 314.1

(51) Int. Cl. A61B 6/00

(52) U.S. Cl. 600/431

(57) ABSTRACT

The invention relates to marking clips for an area of the inside wall of the stomach or intestine. These are characterized in particular by the fact that damaged or treated areas of the inside wall of the stomach or intestine can be easily identified and in this way can be easily found again. For this purpose, a housing accommodates at least one lighting means interconnected with a primary element via a contactlessly actuatable switch, wherein the primary element housing made of metal is at the same time an object that can easily be assigned to the marking clip. Moreover, the housing is connected to a body in the form of a clamp made of a resorbable material for securing to the inside wall of the stomach or intestine, wherein either the clamping jaws of the clamp are connected to each other via at least one spring element, which presses the clamping jaws onto each other and at the same time is a component part of the body, or at least one clamping ring is arranged on the clamping jaws of the clamp.

The invention concerns marking clips for an area of the inside wall of the stomach or intestine.

EP 1 561 420 A2 discloses a system for identification of injuries by means of a marker that is emitting light beams. The marker is placed onto the inner wall of an injured organ by means of an endoscope. The light beams of this marker impinge on the organ wall to which the marker is attached. The injury is in this way marked and can be treated by means of further endoscopes from the exterior. The marker is embodied as a sleeve and has securing means for the inner wall of the organ. After the injury has been treated, the marker must be removed again from the exterior.

The printed publication DE 19630 324 A1 (surgical instrument) discloses a surgical instrument comprised of an elongate flexible actuating cable with a hose-shaped cable sleeve and a cable core that is slidably guided in longitudinal direction within the cable sleeve. By means of it, a marking clip can also be moved into position. The marking clip comprises in this connection forceps with two forceps arms that are springily pretensioned in their open position. The marking clip is comprised in this connection of this forceps-like device. An illumination means is not provided. Removal must again be done by a special intervention.

The printed publication WO 2004/043219 A2 (bioabsorbable marker having external anchoring means) discloses inter alia also markers which can be comprised of a resorbable. These markers are however simple bodies that must be positioned by means of external hooks as fastening means. In this connection, the tissue to be marked is penetrated. Illumination means are not provided in these markers. Moreover, for removal of the fastening means, again a special intervention is required because they are fixedly secured within the tissue.

The printed publication WO 03/093338 A2 (biodegradable polymer for marking tissue and sealing tracts) discloses biologically degradable clips of polymer. They can have very different configurations. They are however not provided with illumination means. Metallic objects are not part of these clips. Therefore, they are biologically degradable but they cannot be easily found in case of a required subsequent control.

In the printed publication US 2004/0015229 A1 (vascular stent with radiopaque markers) stents with markers are disclosed. They are of an annular configuration and are loosely positioned on fingers. The latter are at the same time components of the stent. These markers also have no illumination means. Moreover, they are components of the stents; the placement is thus ensured by the inserted stent.

It is an object of the invention as disclosed in claim 1 to mark a changed or treated area of the inner wall of the stomach or intestine for easy and simple retrieval.

This object is solved by the features listed in claim 1.

The marking clips for an area of the inner wall of the stomach or intestine are characterized in particular in that changed or treated areas of the inner wall of the stomach or intestine can be easily marked and are therefore easily retrievable.

For this purpose, in a housing at least one illumination means is arranged that is connected by means of a contactless actuatable switch with a primary cell wherein the primarily cell housing comprised of metal is at the same time an object that is easily assignable to the marking clip.

Moreover, the housing is connected to a body in the form of a clamp of a resorbable material for attachment on the inner wall of the stomach or intestine, wherein either the clamping jaws of the clamp are connected by means of at least one spring element that forces the clamping jaws against each other and is at the same time a component of the body or at least one clamping ring is arranged on the clamping jaws of the clamp.

By preventive medical examinations in connection with increasing spread of endoscopic examinations carcinomas of the gastrointestinal tract can be recognized often at a very early stage. Frequently, pre-stages of the carcinomas are manifested as polyps or adenomas that can be endoscopically removed. In some cases, when the histological results are known, a carcinoma in situ or a high-grade intraepithelial neoplasia is diagnosed. However, the removal location are then usually already invisibly healed and the actual indicated post resection, meaning a segment resection or full wall excision, are hardly possible anymore. Also, tumors that have not been removed, that are very small and raise biotically the suspicion of a carcinoma and therefore must be subjected to an operation, often represent intraoperative problems in the search.

In most cases it is essential to perform during operation a colonoscopy or gastroscopy by the “rendezvous” method. These intraoperative endoscopies are however complex as well as time consuming in regard to the course of the operation and also cause gas insufflation in the stomach lumen or intestinal lumen which causes the stomach or intestine to expand exorbitantly and in particular in case of laparoscopies is immensely disruptive. The release of the gas after the endoscopy is usually achieved only incompletely so that the stomach or the intestine after the endoscopy is in an unnaturally expanded and pretensioned state. In this state, often anastomoses or staple sutures after resection of the diseased stomach or intestinal section must be carried out. Because of the pretension and expansion the tissue is in a bad blood circulation state and in an unnatural initial position. This favors anastomosis leakage and anastomosis insufficiencies.

For marking the mentioned locations of the stomach or the intestine, the marking clip can be used advantageously. The body of resorbable material with the housing is attached to the inner wall of the stomach or intestine. The material is innocuous as well as tissue-compatible and is resorbed in approximately 2 to 3 months. The marking clip is positioned by a clip placement device during the first endoscopy on the inner wall. When the histological results require a subsequent operation, the latter will be carried out most likely in a time frame of approximately 2 to 4 weeks. During this time the marking clip remains at the location of the inner wall of the stomach or intestine. The illumination means is then not connected to the energy source so that it does not illuminate.

During the operation the illumination means is connected by means of the switch with the energy source. The illumination means emits the light beams into the intestine or stomach and can therefore be easily detected intraoperatively. No additional means which would impair the operation are required. The affected piece of stomach or intestine and the marking clip are removed. Switching on the illumination means is done contactless, for example, inductively, by means of a magnetic field or a sender for electromagnetic radiation.

Should no subsequent operation be performed, the body of the marking clip will dissolve after approximately 2 to 3 months. The housing with the illumination means, the switch, and the energy source are excreted via naturalis through the gastrointestinal tract.

The clamp has at least two clamping jaws between which an area of the inner wall of the stomach or intestine is clamped. In this way, the marking clip is safely secured on the location to be marked of the inner wall of the stomach or intestine.

The clamping jaws of the clamp are connected for this purpose by at least one spring element with each other such that the clamping jaws are pushed against each other. Moreover, the spring element is advantageously at the same time a component of the body. The resorbable material itself may be the spring element. Moreover, a separate spring element may also be provided with the resorbable material such that upon dissolving of the clamping jaws of resorbable material the spring element will detach from the inner wall of the stomach or intestine and will be excreted naturally. Of course, the marking clip or the separate spring element or parts thereof may also be manually removable.

In a further variant, on the clamping jaws of the clamp at least one clamping ring can be arranged. The clamping ring secures a fixed position of the clamping jaws with the area of the inner wall of the stomach or intestine arranged therebetween. The clamping ring in particular can also be moved advantageously on the clamping jaws so that, depending on the position of the clamping ring, a different spacing of the clamping jaws relative to each other is provided. In this connection, the clamping ring is comprised either of a resorbable or a non-resorbable material. In the latter case, it will not dissolve so that also a thin clamping ring can be used. After dissolution of the clamping jaws, the clamping ring is naturally excreted.

The primary cell housing is comprised advantageously of a metal. In this way, the marking clip can be easily assigned by x-ray examination of the abdominal cavity.

Advantageous embodiments of the invention are disclosed in the claims 2 to 11.

The body of the resorbable material according to the embodiment of claim 2 has at least one rough surface, a surface provided with little barbs, a surface provided with a toothing, or a surface with at least one combination thereof. In this way, the marking clip can be attached easily and safely on the inner wall of the stomach or intestine.

At least one of the surfaces of the clamping jaws of the clamp that face each other have according to the embodiment of claim 3 at least one tooth or a row of teeth. When placing the marking dip, it will hook on the inner wall of the stomach or intestine so that a secure holding action of the marking clip is ensured.

Favorably, according to the embodiment of claim 4, the body is made of resorbable material of an organic polymer either as a biopolymer or chemically modified polymer. In this way, it is ensured that the body will dissolve after approximately 2 to 3 months.

According to the embodiment of claim 5 advantageously the illumination means is arranged in the housing or the illumination means is a component of the housing. The housing is comprised in the first variant at least partially of a material transparent for the light beams of the illumination means so that the light beams of the illumination means can reach the exterior.

Favorably, the housing according to the embodiment of claim 6 is comprised of plastic material. Such a housing can be easily produced.

The illumination means according to the embodiment of claim 7 is a luminescent diode that emits blue light. This color is providing the best contrast relative to tissue. Moreover, the illumination means can also be seen well by surgeons with red/green color blindness.

The switch according to the embodiment of claim 8 is advantageously a switch that can be actuated by means of electromagnetic radiation or a magnetic field.

The contactless actuatable switch, the energy source, and the illumination means according to the embodiment of claim 9 are arranged on a component carrier and are connected by conductors. In this way, a compact component group is provided that is easily positionable in the housing or can be easily provided with the housing.

The contactless actuatable switch, the energy source, and the illumination means are located according to the embodiment of claim 10 advantageously in a cured resin body as a housing. In this way, this unit is a compact component group that for formation of the marking clip is connected easily with the body of resorbable material.

The housing with the contactless actuatable switch and the energy source as well as the illumination means according to the embodiment of claim 11 is connected by an adhesive connection with the body of the resorbable material. In this way, a simply realized marking dip is provided. The marking clips can therefore be provided easily as a mass-produced product.

One embodiment of the invention is illustrated in the drawings in a basic illustration, respectively, and will be explained in the following in more detail.

It is shown in:

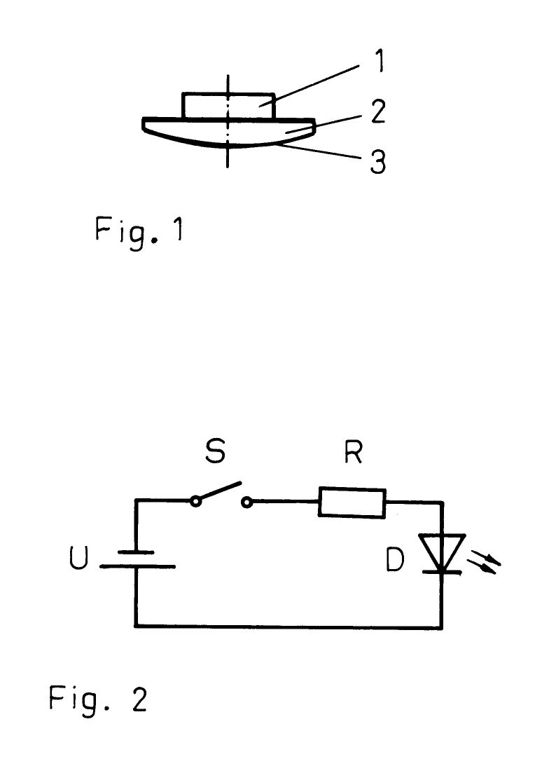

FIG. 1 a marking clip for an area of the inner wall of the stomach or intestine;

FIG. 2 a circuit diagram for a marking clip;

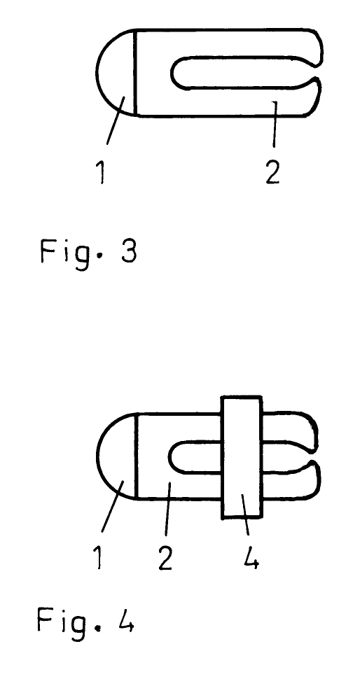

FIG. 3 a marking clip as a clamp; and

FIG. 4 a marking clip as a clamp with a clamping ring.

The marking clip for an area of the inner wall of the stomach or intestine is comprised substantially of a housing 1 with a switch S, an energy source and an illumination means and a body 2 at least partially of a resorbable material.

FIG. 1 shows a marking clip for an area of the inner wall of the stomach or intestine in a basic illustration.

On a component carrier, the energy source as primary cells in the form of a battery as a voltage source U supplying electrical power, a contactless actuatable switch S, a series resistor R as a current source, a luminescent diode D as an illumination means are arranged and connected to each other by means of conductors so that these elements together with the actuatable switch S represent an electric circuit. This component carrier is arranged in the housing 1. For this purpose, this populated component carrier is within a cured resin body as a housing.

FIG. 2 shows a circuit diagram for the marking clip.

The luminescent diode D emits blue light when the electric circuit is closed. Moreover, it is arranged either in the housing 1 or it is a component of the housing 1. In the first variant, the housing is comprised of material that is transparent for the blue light of the luminescent diode D. In the second variant the housing 1 encloses the luminescent diode.

The battery is enclosed by a metal jacket so that it is at the same time the object that is comprised of metal as a component of the marking clip.

The switch S is a switch S that is actuatable either by electromagnetic radiation or a magnetic field.

Upon action of the magnetic field on the switch S, its switching contacts are connected with each other so that the electric circuit with the luminescent diode D is closed and the latter emits blue light. In this connection, the magnet can be advantageously provided on the endoscope at the same time.

The housing 1 is connected with the body 2 of resorbable material for attachment on the inner wall of the stomach or intestine. This connection is based advantageously on an adhesive connection. The body 2 of resorbable material has at least one surface 3 that is rough, has barbs or a toothing or at least a combination thereof.

In one embodiment of the body 2, the body can be at least a part of a clamp for the inner wall of the stomach or intestine.

FIG. 3 shows a marking clip as a clamp in a basic illustration.

The surfaces of the clamping jaws of the clamp that face each other have at least one tooth or a row of teeth.

In one variant the clamping jaws of the clamp are connected to each other by at least one spring element that forces the clamping jaws against each other. The spring element is at the same time a component of the body.

In another variant, on the clamping jaws of the clamp at least one clamping ring 4 is arranged. The clamping ring 4 is comprised of the resorbable material or a material that cannot be resorbed. The clamping jaws of the clamp can moreover also be designed such that the cross-section of the clamping jaws in the longitudinal direction of the clamp either continuously or steadily increases or decrease. With a movement of the clamping ring on the body, the clamping jaws are thus moved toward each other. In this way, the clamp is attached on the inner wall of the stomach or intestine. FIG. 4 shows a marking clip as a clamp with a clamping ring 4 in a basic illustration.

The body 2 of the resorbable material is comprised of an organic polymer either as a biopolymer or chemically modified polymer.

What is claimed is:

1. A marking clip for an area of the inner wall of the stomach or intestine, the marking clip comprising:
a housing with at least one illumination means that is connected by a contactless actuatable switch in the housing with a primary cell in the housing, wherein the primary cell has a metal housing that is detectable by x-ray radiation of the abdominal cavity for locating the marking clip,
a body connected to the housing, wherein the body is a clamp of a resorbable material for attachment on the inner wall of the stomach or intestine, wherein the clamp has clamping jaws that are either connected to each other by at least one spring element that forces the clamping jaws against each other and is a component of the body or at least one clamping ring is provided that is arranged on the clamping jaws of the clamp.

2. The marking clip according to claim 1, wherein the body of the resorbable material has at least one rough surface, a surface provided with barbs, a surface provided with a toothing, or a surface with at least one combination thereof.

3. The marking clip according to claim 1, wherein at least one of the surfaces of the clamping jaws that face each other has at least one tooth or a row of teeth.

4. The marking clip according to claim 1, wherein the body of the resorbable material is comprised of an organic polymer that is a biopolymer or a chemically modified polymer.

5. The marking clip according to claim 1, wherein the at least one illumination means is arranged in the housing or at least one illumination means is a component of the housing.

6. The marking clip according to claim 1, wherein the housing is comprised of a plastic material.

7. The marking clip according to claim 1, wherein the at least one illumination means is a luminescent diode emitting a blue light.

8. The marking clip according to claim 1, wherein the contactless actuatable switch is actuatable by electromagnetic radiation or by a magnetic field.

9. The marking clip according to claim 1, wherein the contactless actuatable switch, the energy source, and the at least one illumination means are arranged on a component carrier and are connected to each other by conductors.

10. The marking clip according to claim 1, wherein the housing is a cured resin body and the contactless actuatable switch, the energy source, and the at least one illumination means are located in the cured resin body.

11. The marking clip according to claim 1, wherein the housing containing the contactless actuatable switch, the energy source and the at least one illumination means is connected by an adhesive connection to the body.
